(19) United States
(12) Patent Application Publication
(10) Pub. No.: US 2014/0074086 A1
(43) Pub. Date: Mar. 13, 2014

(54) Patient Warming/Electro-surgical Grounding Pad

(71) Applicants: Mary Elizabeth MacIntyre-Ellis, Seattle, WA (US); Kent Douglas Ellis, Seattle, WA (US)

(72) Inventors: Mary Elizabeth MacIntyre-Ellis, Seattle, WA (US); Kent Douglas Ellis, Seattle, WA (US)

(73) Assignee: Pintler Medical, Seattle, WA (US)

(21) Appl. No.: 13/610,695

(22) Filed: Sep. 11, 2012

(51) Int. Cl. A61B 18/16; A61F 7/00

(52) U.S. Cl. CPC A61B 18/16; A61F 7/007
USPC 606/35; 607/96

(57) ABSTRACT

A surgical operating room table pad that functions in multiple capacities such as a dielectric capacitor for grounding the electro-surgical current from cautery device and heating the patient contact surface for the maintenance of patient normal-thermia.

The “capacitor/dielectric” is designed to allow the electrosurgical current introduced to the patient to return back to the electrosurgical generator for patient and surgical staff safety.

The temperature of the patient's body is constantly measured by a series of RTD Thermister sensors and a resistive device is utilized for thermal input.

### CROSS-REFERENCE APPLICATION(S)

U.S. Patents Documents

| | | |
|---|---|---|
| 4,310,745 | January 1982 | Bender |
| 4,736,088 | April 1988 | Bart |
| 47/888,417 | November 1988 | Graflind |
| 4,825,868 | May 1989 | Susa |
| 5,138,138 | August 1992 | Theilacker et al. |
| 5,324,911 | June 1994 | Cranston et al. |
| 5,371,340 | December 1994 | Stanfield |
| 5,385,529 | January 1995 | Koch |
| 5,451,747 | September 1995 | Sullivan et al. |
| 5,516,189 | May 1996 | Ligeras |
| 5,720,774 | February 1998 | Glucksman |
| 5,785,716 | July 1998 | Bayron et al. |
| 5,881,410 | March 1999 | Yamada |
| 5,932,129 | August 1999 | Hyatt |
| 5,948,303 | September 1999 | Larson |
| 6,006,136 | December 1999 | Glucksman |
| 6,083,221 | July 2000 | Fleenor et al, |
| 6,582,456 | June 2003 | Hand et al. |
| 6,653,607 | November 2003 | Ellis et al. |
| 6,924,467 | August 2005 | Ellis et al. |
| 6,933,469 | August 2005 | Ellis et al. |
| 6,967,309 | November 2005 | Wyatt et al. |
| 7,176,419 | February 2007 | Ellis et al |
| 7,196,289 | March 2007 | Ellis et al. |

Applications

| | | |
|---|---|---|
| 20080255641 | Oct. 2008 | Ellis |

Provisional application to be abandoned: 61,465,898
Application Number: 13/506,053, dtd Mar. 24, 2012

### OTHER REFERENCES

a. Specifications Manual for National Hospital Inpatient Quality Measures, SCIP-10 Version 3.2
b. A Randomized Comparison of Intraoperative PerfecTemp and Forced Air Warming During Open Abdominal Surgery, Anesthesis-Analgesia, October 2011. Authors: Egan, Bernstein, Redd, Ali, Paul, Yang, Sessler
c. Estimation of mean body temperature from mean skin and core temperature. Anesthesia Journal December 2006. Authors Lenhardt, Sessler
d. Mild Intraoperative Hypothermia prolongs postanesthetic recover. Anesthesia Journal December 1997. Authors Lenhardt, Marker, Goll, Tschernich, Kurz, Sessler, Narzt, Lackner
e. Perioperative normothermia to reduce incidence of surgical-wound infection and shorten hospitalization. Study of Wound Infection and Temperature Group. NCBI, NLM.gov, published in PubMed and New England Journal of Medicine May 9, 1996. Authors Kurz, Sessler, Lenhardt
f. Warming by resistive heating maintains perioperative normothermia as well as forced air heating, British Journal of Anesthesia, January 2003. Authors: Matsuzaki, Matsukawa, Ohki, Yamamoto, Nakamura, Oshibuchi.
g. Perioperative maintenance of normothermia reduces the incidence of morbid cardiac events. Journal of the American Medical Association, 1997. Authors: Frank, Fleisher, Breslow & et al.
h. Effects of preoperative warming on the incidence of wound infection after clean surgery, a randomized controlled trial. The Lancet, British Journal of Medicine, September 2001. Authors: Melling, Ali, Scott, Leaper.
i. IEC60601-1, 3rd Edition, Electrical Safety Standards
j. IEC60601-1-2 EMC Standards
l. IEC80601-2-35 Heated Patient Surface Safety Standards

### TECHNICAL FIELD

The following disclosure relates generally to personal warming systems that aid in the maintenance of patient normal thermia during the Perioperative experience. The disclosure relates to apparatuses involved with electrosurgical grounding of patients during surgical, diagnostics and invasive procedures.

### BACKGROUND

Patient warming: maintaining a patient's body temperature is an important part of the surgical experience. Studies have shown that patients with a +36 C post operative core body temperatures experience reduced post operative infection rates and other life threatening complications. Surgical patients who experience core temperatures −36 C exhibit twice (2×) the post operative infection events (Lancet Study, Sep. 15, 2001, Volume 358, Number 9285, study group: 421 surgical patients). The outcome of the study have been identified as a land mark for understanding the relationship of patient warming and post operative patient infection events.

“Specifications Manual for National Hospital Inpatient Quality Measures, dated Oct. 1, 2010, under the Measured Information Form has adopted a program identified as SCIP-10, Version 3.2. This measure was adopted by Center for Medicare Services (CMS) division of Health and Human Services (HHS) and the Joint Commission of Hospital Accreditation (JACHO). The measure specifically requires surgical patients to be actively warmed to one body temperature equal or greater than 96.8 F/36 C and be recorded within 30 minutes prior to surgery and/or 15 minutes immediately after anesthesia time.

Conventional modes of patient warming exist, Augustine U.S. Pat. No. 6,210,428 has exhibited the use of Forced Air into a disposable covering positioned upon the patient as a successful method of patient warming.

Another method is “warm water circulating blanket” beneath the patient. This method was developed and adopted in the 1960's. It remains today as a patient cooling product versus warming.

Ellis, U.S. Pat. No. 6,653,607 introduced resistant material warming in 2002 and studies have shown this method of patient warming to be successful in many procedures.

Use of electrosurgical tools and products during surgery is for the purpose of cutting or coagulating vessels. Physician use of electrosurgical generators & cautery pencils requires measures for patient safety. Every use of an electrosurgical tool i.e. the cautery pencil, requires that the current return to the electro-surgical generator from the cautery pencil or cautery device. This power in/power return is called the electrosurgical loop. The return of current is managed and measured to prevent a patient from experiencing an electrosurgical burn, the loop system is for patient safety. Fleenor U.S. Pat. Nos. 6,083,221/6,053,910 and subsequent filings in the name of Fleenor address the application and use of a Dielectric Capacitor with electro-surgical devices.

This utility design allows a single product, the operating room surgical pad to fulfill two (2) separate functions in the care of a patient during surgery: 1) maintenance of norma-thermia with a resistive heating design and 2) receiver and housing unit for a reusable capacitor device to receive current from the cautery pencil and return it to the electrosurgical generator. Meaningful use: reduction in medical cost, medical waste, time, utility use for all patients, reduced surgical infection rates and patient safety.

### DESCRIPTION OF THE DRAWINGS

Drawing #1, overview of a heated operating room table pad positioned upon a standard surgical table. Noted in the background is the 1) power unit positioned/mounted to an IV pole in proximity of the operating room table. 2) The heated operating room table pad is connected to the power control unit via a ribbon/cable. 3) The power control unit is connected via cable to the patient monitoring system or temperature module. 4) The electro-surgical generator connected via cable to the Black Fiber Carbon Capacitor electro-surgical grounding device positioned within the heated operating room table pad. A dotted line exhibits the position/location of the Black Carbon Fiber Electro Surgery Capacitor/Grounding device element being positioned beneath the pad's covering or outer coating. 5) The data and/or wireless connection to the patient monitoring system.

Drawing #2, overview of a heated operating room table pad positioned upon a standard surgical table. Noted is the optional positioning of the power unit w/microprocessor within the confines of the surgical table support column. A cut out is provided as an example of the hand controller w/optional patient warming controls accompanying the patient positioning controls.

Drawing #3, overview of the operating table pad exhibiting the temperature sheath/channel/pocket positioned center and in a topical position upon the pad's cover. Dotted line represents the sub-cover position of a resistive heating element and/or electro-surgical capacitor/grounding pad in relationship to the pad and the channel/sheath/pocket. Exhibited are two (2) cables/ribbons: (1) Heating element w/thermisters to control unit. 2) Black Carbon Fiber Electro Surgical Capacitor/Grounding to the electro-surgical generator.

Drawing #4, side view of the operating table pad exhibiting a vinyl or spray coat coating/cover with cables/ribbons exiting the pad. Location and approximate position of the temperature measurement system cable/ribbon and Electro Surgical Capacitor/Grounding within the pad.

Drawing #5, overhead view of the resistive heating element within the pad. A detailed cut out reveals the element to be carbon embossed on the substrate and the open space between each element. Common attachment to a copper buss bar is shown.

Drawing #6, view of the resistive heating element revealing the site and appearance of the connection site and apparatus to be applied to the resistive element. Revealed in the drawing is the optional placement of the temperature sensor system in direct contact with the resistive heating element.

Drawing #7, mechanical drawings of the two types of temperature sensor systems: design (A) reveals the Cicoil design utilizing a silicone ribbon with thermister mounted at specific sites and the ribbon assembled to directly connect to the power supply. Design (B) reveals the Flex Circuit design, functioning similar to the design (A).

Drawing #8, side pad view exhibiting the two variances of design for the ribbon/cables to enter/exit the pad with the variances of covering designs, spray coat vs. sewn slip on vinyl cover.

Drawing #9, overhead view and side view of the Black Carbon Fiber Electro-Surgical Grounding device positioned within the parameter of the pad and the pad's design. Exhibited is a preferred location for the connecting cable to exit the pad.

Drawing #10, view of the two optional ribbons/cables w/thermisters and clearly printed with an identity that is Xray visible.

Drawing #11, views of the power unit and membrane. Revealed are the layout of the control membrane, informational LED's, temperature control selections. Identified are the positions and locations for external cable connections.

### DETAILED DESCRIPTION

The following disclosure describes the operation of various aspects of the patient warming/electro-surgical grounding system. The system is designed for use during surgical and diagnostic procedures or events that require the maintenance of patient normal-thermia and/or use as a electrosurgical grounding device for the electrosurgical cautery pencil/device. The disclosure presents the novelty to maintain patient normal-thermia without a disposable product and at the same time offering to the surgeon a secondary function of the pad to function as a electro-surgical grounding capacitor. A novel single component in the operating rooms to replace other single use devices.

In one embodiment the heated/grounding operating table pad is positioned upon a operating room table in the ON state. The operator has the option to select a skin temperature ranging from 37 C to 42 C. The operator selects the desired skin temperature and the control unit microprocessor responds and with a “green” LED light and audible cheep confirming the selection. With temperature selection input the CPU's microprocessor software reviews the operator's input and compares the data from the pad's temperature measurement system, power is adjusted to the resistive element.

In an optional “built in” embodiment the CPU board, power supply and connectors can be incorporated into the frame and/or design of the operating table platform. The hand controller of the operating table would be modified to reflect the operator's controls for the patient warming feature.

In another embodiment the operator can elect to connect the electro-surgical grounding device cable to the electro-surgical generator and thus have the REM loop completed and offer the patient a safe environment the use of RF current for surgical cutting or coagulation from the electrosurgical generator, i.e. Megadyne, ConMed, Valley Lab and others.

Due to concerns and variance of practices the use of two outer cover embodiments is offered. The vinyl sewn has a history of utilization, the embodiment utilizing a spray vinyl cover is an innovation and offers a variance in cleaning, infection control barrier and pressure reduction practices. The two embodiments do not change the design or layering of the pad. Both covers function is a similar manner in this application.

In the warming embodiment the patient's supine skin temperature is measured via a line of sensors positioned and secured within the pad's protective sheath/channel/pocket or beneath the spray vinyl covering. When the temperature measurement system is topically positioned it is center line of the operating room pad and actively measures the patient's skin. In another embodiment the sensors can be positioned upon or beneath the heating element and control the thermal input with direct measurement of the resistive element's temperature.

RTD sensors utilized for temperature measurement are uniquely qualified due to their narrow resistance range. These thermisters are positioned within the cover and in a technique of soldered attached to a ribbon cable or a flex circuit ribbon. This ribbon/cable design exhibits a Sub D connector.

In another embodiment the control unit's offers direct connection of a individual temperature RTD sensor to the patient's electronic monitoring & record. A connection port is offered for a cable connection to the patient anesthesia monitoring system. In this embodiment the temperature data collected can verify that the patient was actively warmed and recorded for compliance with patient care requirements.

In another embodiment the importance of safety cannot be understated. In this embodiment the CPU microprocessor software's method does not allow nor convey DC electrical power to the resistive heating element if an unsafe temperature is reported by any thermisters of the temperature measurement system. Immediate patient safety occurs when a surface temperature above 42 C or a temperature variance of 2 C between measured temperature and the operator's selection has been revealed by any thermister of the temperature measurement system.

Overall the system is quite simple, a operating table pad that heats and offers electro-surgical grounding surface to couple with the electrosurgical generator. Utilization of RTD sensors to accurately measure temperature on the pad's top surface or at the heating element. Availability of the carbon fiber electrosurgical grounding device with minimum effort from staff. A power unit either IV pole mounted or housed within the body of the OR table structural design. CPU Board hardware/software that utilizes data input from the RTD sensors to determine power input to a resistive device of unique design. Cables connecting the grounding device to the electro-surgical generator.

### Review of Drawings:

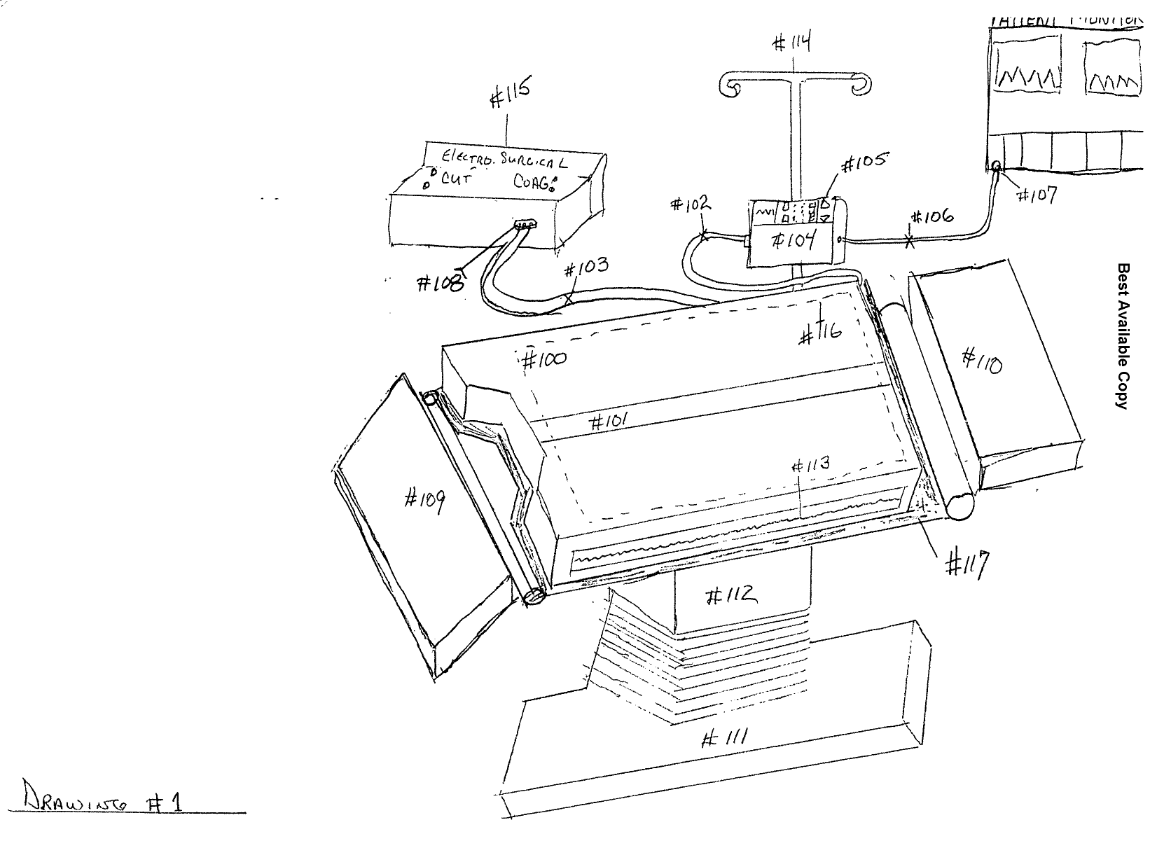

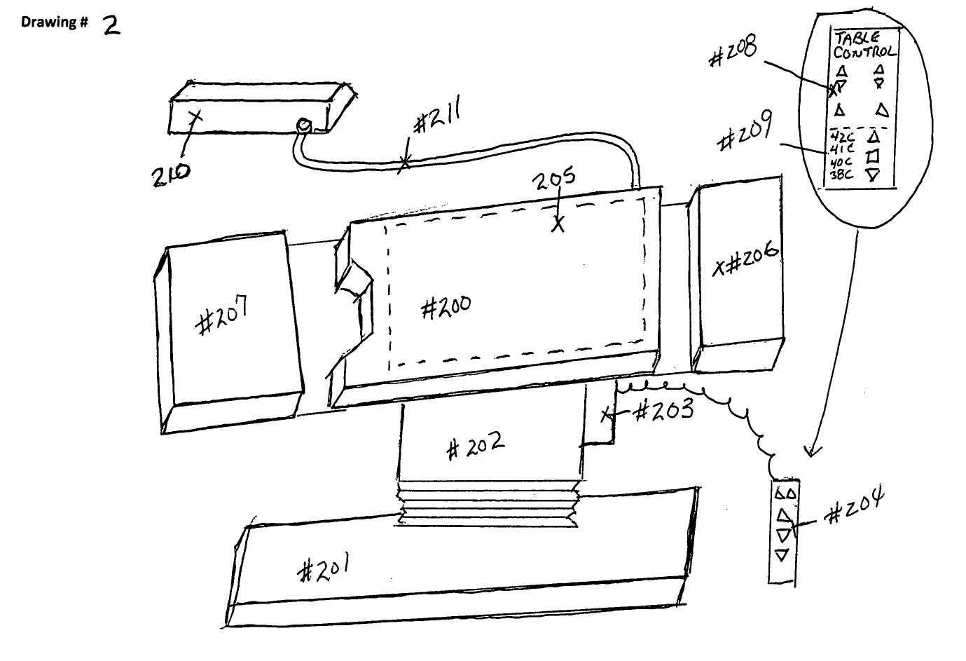

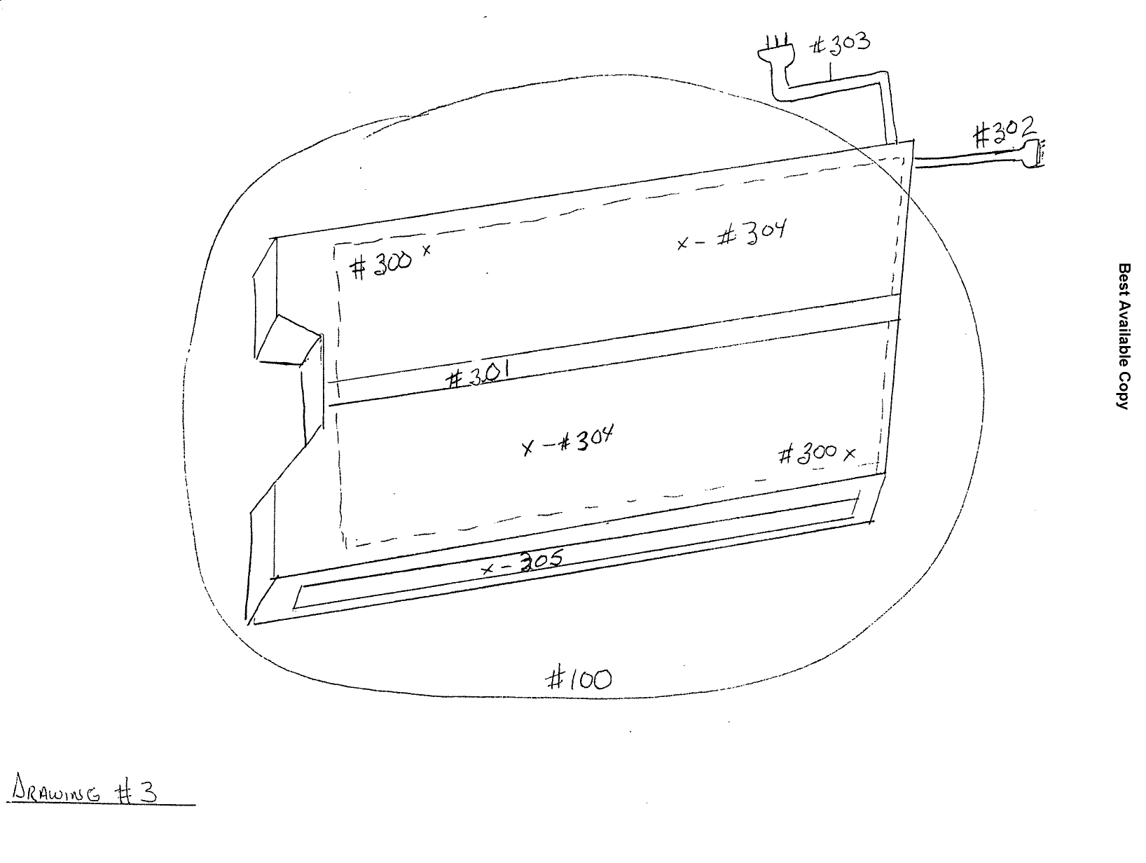

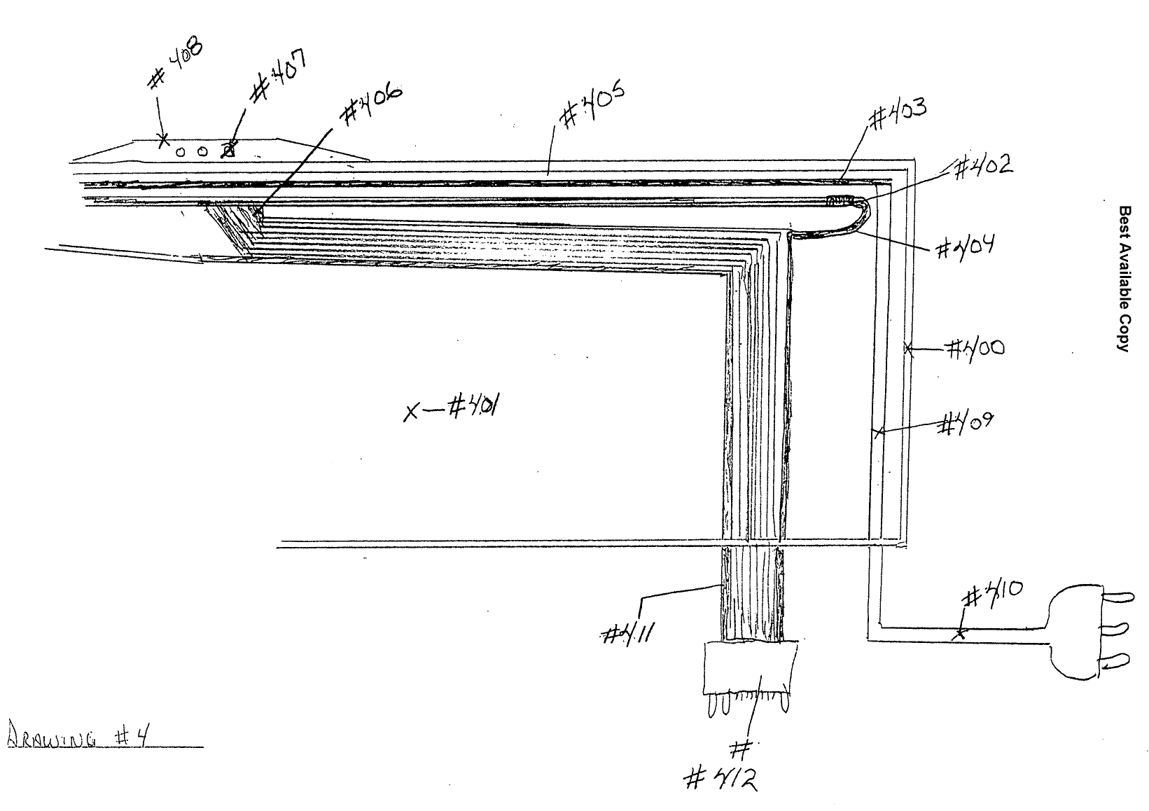

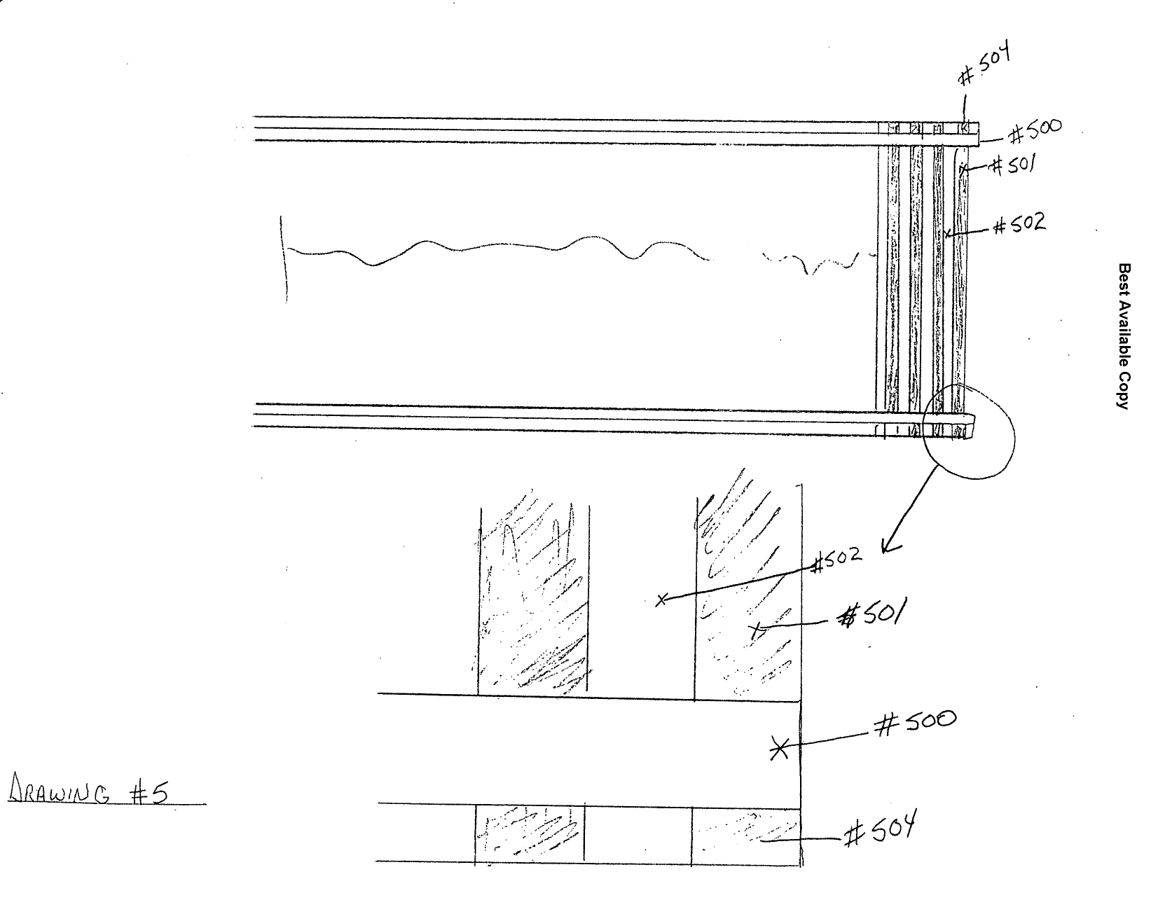

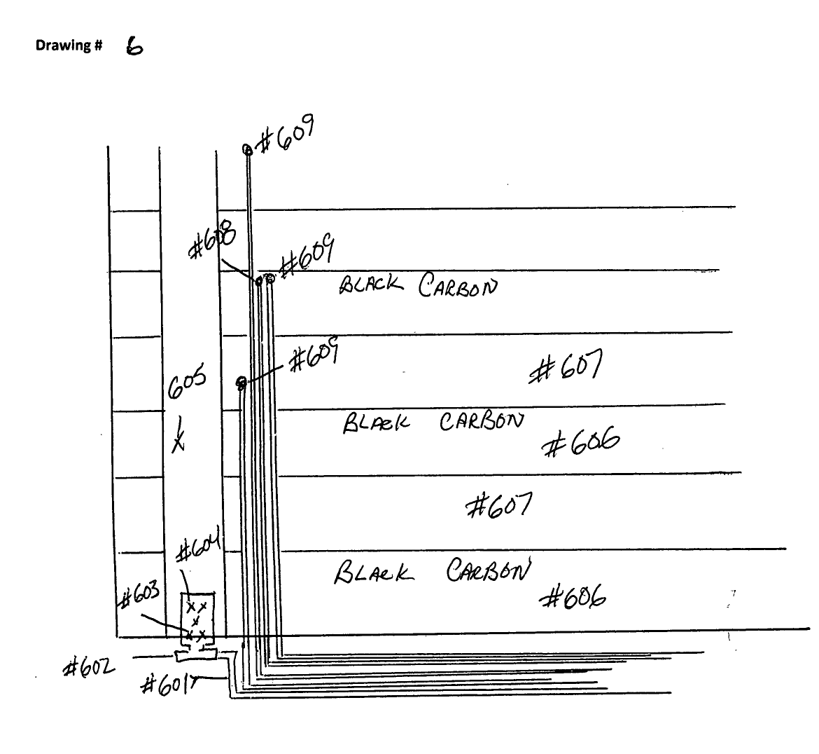

In the figures, identical reference numbers identify noted area. To facilitate the discussion and review of any particular element, the initial digit (s) will reference the figure number and the subsequent digits will be referenced as identified in the figure. I.e. FIG. 1, all reference items will be first identified as 1XX.

Drawing #1, standoff view of an operating room table with one part of the invention (the pad) positioned upon the platform or surgical table. Exhibited is the mounting of the control unit to an IV pole and the approximate location of the electro-surgical generator for use with the electro-surgical cautery devices.

The operating table is capable of raising or lowering and may exhibit a number of different cushions or pads for positioning of the feet and head. Pads utilization depends on surgical procedure, positioning and patient's weight/height ratio. 100 exhibits the heated/grounding operating room pad positioned upon a standard operating room table. 101 exhibits the temperature measurement pocket/channel/sleeve positioned and adhered to the top of the operating room table pad cover. 102 exhibits the cable/ribbon joining the operating room pad and the operator's control unit. 103 exhibits the cable joining the capacitor/grounding device within the pad to 115 the electro-surgical generator. 104 exhibits the control unit mounted to an IV pole. 105 exhibits the operator's control membrane, positioned upon the control unit. 106 exhibits the cable joining the control unit to the patient monitoring system. 107 exhibits the connector joining of the cable from the control unit to the patient monitoring system. 108 exhibits the cable joining the capacitor/grounding device within the pad mating to 115 the electro surgical generator. 109 exhibits the operating table foot pad. 110 exhibits the operating table head pad. 111 exhibits the base of the OR table. 112 the operating table height adjustment column. 113 lateral view of the operating pad's side zipper location. 114 standard operating room IV pole. 115 a general impression of the position of an Electro-surgical generator in the operating room. 116 envisioned position of the electro-surgical grounding device beneath the pad's cover (s).

Drawing #2, 200 exhibits the heating/grounding pad positioned upon an operating room table. 206 & 207 are identified as table pads for the head and foot sections. 201 & 202 a operating table platform and column with a modification to compliment the installation, addition or modification of the control process and needed electrical components to operate the resistive heating technology of the pad. 204 exhibits a projected operating table hand controller modified to include the selection of temperature or heating control. 208 & 209 exhibit a projected design of the operating table controller w/heating adjustments. 211 exhibits the connection cable to the electro-surgical generator. 210 is an example of an electro-surgical generator.

Drawing #3, exhibits an overhead view of the surgical torso pad. 300 is a pad with a sewn vinyl cover. 301 exhibits the temperature channel attached to the overall cover. 302 identifies the ribbon/cable exiting the pad for connection to the control unit. 303 identifies the exiting electro-surgical grounding cable from the pad for connection to the electro-surgical generator. 304 reveals/identifies the approximate position beneath the pad's cover of the electro-surgical capacitor/grounding device. 305 exhibits a side zipper on the pad with a protective flap.

Drawing #4, is a lateral view from the top end of the surgical table pad, width is 20″. 400 exhibits the outer conductive vinyl blend cover. 401 exhibits the base or foundation foam. 402 exhibits the location of the laminated heating element positioned below the outer cover, upon the foam base 401. 403 exhibits the location of the electro-surgical grounding device location upon the heating element. Note components 402 and 403 can be reversed with heating element upon the electro-surgical grounding element. 404 exhibits the 18 AGV wire from the cable/ribbon to the heating element crimps. 405 exhibits the location and relationship of the felt thermal diffuser material. 406 proximity or relationship of the temperature sensor ribbon/cable to the pad's channel/sheath/pocket of the vinyl fabric cover. 407 reflects the sensors within the pad's channel/sheath/pocket. 408 covering fabric of the pad's channel/sheath/pocket. 309 cable connected to the electro-surgical grounding element. 410 cable from the electro-surgical grounding element to the electro-surgical generator. 411 Cable/ribbon exiting the pad. 412 Sub D connector joins the cable/ribbon and the power unit.

Drawing #5, is a overhead view of the resistive element. 500 exhibits a single layer copper conductive bar of roughly 0.5″ in width the length of the heating element. 501 exhibits a carbon resistive material roughly 1″ wide that is repeated every other ¾″ the length of the heating element. The length of the resistive element will vary with the different types or lengths of operating room table pads. 502 open area between resistive elements. 504 exhibits border space between the edge of the element and a copper conductive bar

Drawing #6, exhibits how the Tyco clamp/Flexel connector is contacted securely to the buss bar of the heating element. 600 exhibits the cable/ribbon entry to the pad and split or feeding of sensor ribbon and power wires to the element. 601 exhibits 18 AGV separating from the cable/ribbon and leading to contact with the crimped connectors attached to the buss bars. 602 exhibits the 18 AGV wiring crimped to the Tyco/Flexel connector. 603 exhibits the clamp fastened through the outer plastic coating and making contact at multiple locations as the clamp is crimped through the coating, the copper buss bar and Mylar substrate. 604 contact is 5 perforations sites per Tyco clamp/connector. 605 copper buss bar element at each lateral side of the heating element. 606 resistive carbon element. 607 open space between carbon bars. 608 ribbon positioned upon the lateral edge of the heating element.

Drawing #7, overhead view of the Cicoil and Flex Circuit temperature systems w/thermister (s) mounted and connector attached. 700 exhibits the a length of 84″ of Cicoil ribbon and/or Flex Circuit ribbon w/connector. 701 exhibits the separation of 18 AGV wiring from the Cicoil or the Flex Circuit destine for attachment to the heating element Tyco crimps. 702 exhibits the position of thermister (s) upon the Cicoil ribbon and/or Flex Circuit. 703 exhibits the site of the single skin temperature thermister. 704 lateral view of the Cicoil and/or the Flex Circuit ribbon w/2 outer 18 AGV wiring on the perimeter and 8 each 26 AGV wires in the middle section. 705 exhibits a D shaped connector for mating with the control unit. 706 D connector pin alignment for user safety.

Drawing #8 exhibits the pad with both covering materials, a sewn vinyl cover and a spray coat vinyl cover. The view given in this drawing is a lateral view from the head position of the pad. 800 identifies the inner position of the foam base material with both covering materials. 801 identifies the sewn vinyl cover being utilized. 802 identifies the heating element beneath. 803 exhibits the electro surgical grounding element positioned upon the heating element. 804 exhibits the position of the thermal diffusing material. 805 exhibits the relationship of the Igloo cover and 806 exiting electro surgical grounding cable. 807 the ribbon/cable w/connector. 808 identifies the exit site and slit opening of the ribbon/cable from the pad. 809 reveals a back plate used to reinforce the exit site of the ribbon/cable. 810 identifies the spray coat covering over the pads internal components.

Drawing #9, 900 exhibits the patient warming pad w/electro-surgical grounding element positioned in relationship to the pad's overall dimensions. design. 901 exhibits the electron surgical grounding element positioned topical to the base foam. This position could be upon the heating element or beneath the element. This position could be upon the heat diffusion layer or beneath it. 902 site of cable connection to the element. 903 electro-surgical grounding cable exit the pad and connecting to the electro-surgical generator. 804 exhibits either pad cover, sewn vinyl or spray coat vinyl.

Drawing #10, exhibits the temperature measurement systems of the Cicoil product and Flex Circuit with labeling of “sensor-sensor-sensor” repeated in relationship to thermisters. 1000 identifies the two (2) temperature management systems. 1001 exhibits the separation of the 18 AGV wires to the mate with the heating element. 1002 thermister sensors attached to the Cicoil or Flex Circuit. 1003 skin temp thermister. 1004 coating and/or identity label beneath the Cicoil ribbon or Flex Circuit reading sensor-sensor-sensor”

Drawing #11, exhibits a frontal view the control membrane. 1100 exhibits the overall view of the membrane and a dimensional relationship. 1101 exhibits the ON/Standby button. 1102 identifies the Standby LED. 1103 exhibit operator's information LEDs. 1104 represents the vertical rise in temperature selection as the operator elects temperatures in an increase value manner. 1105/1106 exhibits the temperature selection arrows to raise or lower the temperature selection, range of 37 C to 42 C. 1107 temperature gradients LED back lighted that reflect the operator's temperature selection. 1108 exhibits LED lighting areas that advise the operator of operational events or actions required. 1109 alarm mute button. 1110 company labeling site. 1111 exhibits the connection site of the “pass through” patient temperature connector for the connection to the patient monitoring system. 1112 exhibits site of the Sub D connector connection site and location.

### DETAILED DESCRIPTION

The following disclosure describes various aspects of the patient warming system for use in the maintenance of normal-thermia/patient warming and performing as the electro-surgical grounding device for patients experiencing surgery or diagnostic procedures. In one embodiment the patient warming system is made up of a heated operating room pad, the control unit and a joining cable. In another embodiment the pad functions as an electro-surgical grounding device allowing for the current of the electro-surgical cautery to exit the operating table site via a capacitor element such as Black Carbon Fiber or like element located within the confines of the surgical pad. In another embodiment the operator utilizes both capabilities in the care of surgical patient: patient warming and electro-surgical grounding being utilized and functioning in unison during the surgical procedure.

The embodiment of the controller or power unit offers a variety of options. In one embodiment the control/power unit can be in an encasement and designed for positioning upon an IV pole. In another embodiment the power unit could be so assembled as to be a component of the overall operating table design with the elements of temperature control being integrated into the operator's control handle used for adjusting the table's height and angle. In either embodiment, an IV pole mounted control unit or integrated into the tables mechanical design, either pad can be utilized and both functions of warming and grounding are available.

The embodiment as a patient warming device allows the operator a range of temperature selections. Temperature selection is necessary due to variables in patients, procedures and irrigation fluid applications. Temperature selections range from 37 C-42 C, these temperature represent “a safe range” for patient warming. 42C has been determined by healthcare professionals and the FDA as the safe upper maximum range for skin warming of a patient undergoing surgery.

The embodiment of the pad's outer finish is offered in two similar but different coverings. One embodiment is the pad with a sewn vinyl cover and in another the covering material is a spray coat vinyl. The embodiment offers equal protection from fluid invasion, thermal transfer and functionality of the capacitor element for electro-surgical grounding.

In the embodiment of temperature management thermister sensors are securely positioned upon a Cicoil ribbon or a Minco Flex Circuit. In this embodiment many sensors of like Ohms resistance are positioned so as to compliment the human torso in surgical positions. The temperature management system can be position in a variety of sites of the pad, down the center in the identified channel, upon the heating element or beneath the element.

If the operator is electing to X-ray the patient the temperature measurement ribbon or flex circuit when positioned in the center line position is easily identified by the X-ray visible strip identified as sensor-sensor-sensor or other words to aid in identity. When the temperature measurement system is positioned upon or under the heating element site location minimizes the need to identify the temperature management system.

In the embodiment whereas the capacitor electro-surgical grounding device is utilized a separate and protected site is created to allow the cable to transition from the pad to the electro-surgical generator. The positioning of the capacitor grounding device is a capability of either cover material, sewn vinyl or spray coat vinyl.

In the embodiment of cables and connectors to the two optional units, IV pole mount and table integrated cables are identified as wall power to the unit (s), ribbon cable from the pad to the control device, monitoring cable from the device to the patient monitoring system. If the power/controller is of the embodiment of integration into the operating table design the hand held controller which operates the tables positions can exhibit the temperature control selections and information.

FIG. 1 is a view of the heated/electro-surgical grounding operating table pad upon a surgical operating table, the control unit is mounting to an IV pole, the electro-surgical generator is positioned in the surgical suite, and a cable is shown to connect the control unit to the patient monitor. Connection ribbons/cables join the heated operating table pad and control unit. Connection cables join the electro-surgical grounding element to the electro-surgical generator.

From this view the embodiment the heated operating room pad is clearly identified from the other pads utilized surgical positioning, I.e. head and leg pads 109 & 110. 101 illustrates the embodiment of the pad's center/medial (spinal line) and location of the temperature measurement system. 101 is located center of the pad and centered upon the resistive element 400, 500. This position is a point of safety in that the 101 temperature measurement system is directly centered over the resistive element 400, & 500 and thus measuring the highest possible central thermal temperature of the resistive element.

Drawing #1 in accordance with this embodiment the control unit 104 is mounted to an IV pole. In the embodiment the control unit is clamped to the IV pole 114. The operator's membrane of this embodiment allows the selection of desired pad temperatures, 37 C to 42 C as shown in 1100. The operator is advised by “Ready” with LED indicator light 1108 during normal operation. LED's of 1103 advises the operator of abnormal operation. In this embodiment the operator is advised of a failure to join the connector between the pad and power unit 1108. In this embodiment the operator is advised of a temperature measured in excess of 42 C or 2 C beyond the operator's selection as measured by any of the temperature measurement RTD sensors, a visual alarm 1109 is activated. The LED 1108 will remain active when any RTD sensor measures a temperature in excess of 42C and or is in excess by 2 C of the operator's selection. In this embodiment the Warning LED will remain visible until the temperature measured by 800 has fallen below 42 C or less than 2 C of the operator's selection. An example: the operator selects 39 C 1105/1106 and the temperature measurement system 800 measures 41.2 C. The Over-temp/Auto Off LED of 1108 & 1103 will be visible and power is removed from the resistive element 200. An audible alarm activates if the operator attempts ON/Standby or use the device when an unsafe temperature is reported. 1109 allows the operator to mute the audible alarm.

Drawing #2 reflects the power unit and control device being integrated into the mechanical and physical design of the operating room table. 201/202 reflects the mechanical column of the operating room table, 203 reflects the attachment or addition of the heating element power/control unit as a part or addition to the operating table. 204 is a gross projection of the temperature control functions joining the table control device as a single tool for the operator. 205 reflects the electro-surgical capacitor element positioned within this type of operating table design as a component of the pad. 211 reflects the electro-surgical capacitor element's cable exiting the pad separate of the operating table's mechanical design.

Operational instructions are provided to the operator by the control unit membrane 204, 1100. LED's advise the operator when the connector cable/ribbon 102 has not been fully seated with activation of the “Check Connector” 1108. Any resistance measurement failure by an RTD sensor results in a “Service Required” LED of 1108 is activated.

Temperature selection embodiment 204/1105-6 requires an operator's action. The temperature measurement selections 204/1104 reflects operator's objective skin temperature. The microprocessor of #104 regulate electrical current to the heating element 402 to maintain the operator's temperature selection and the heating element's safe performance.

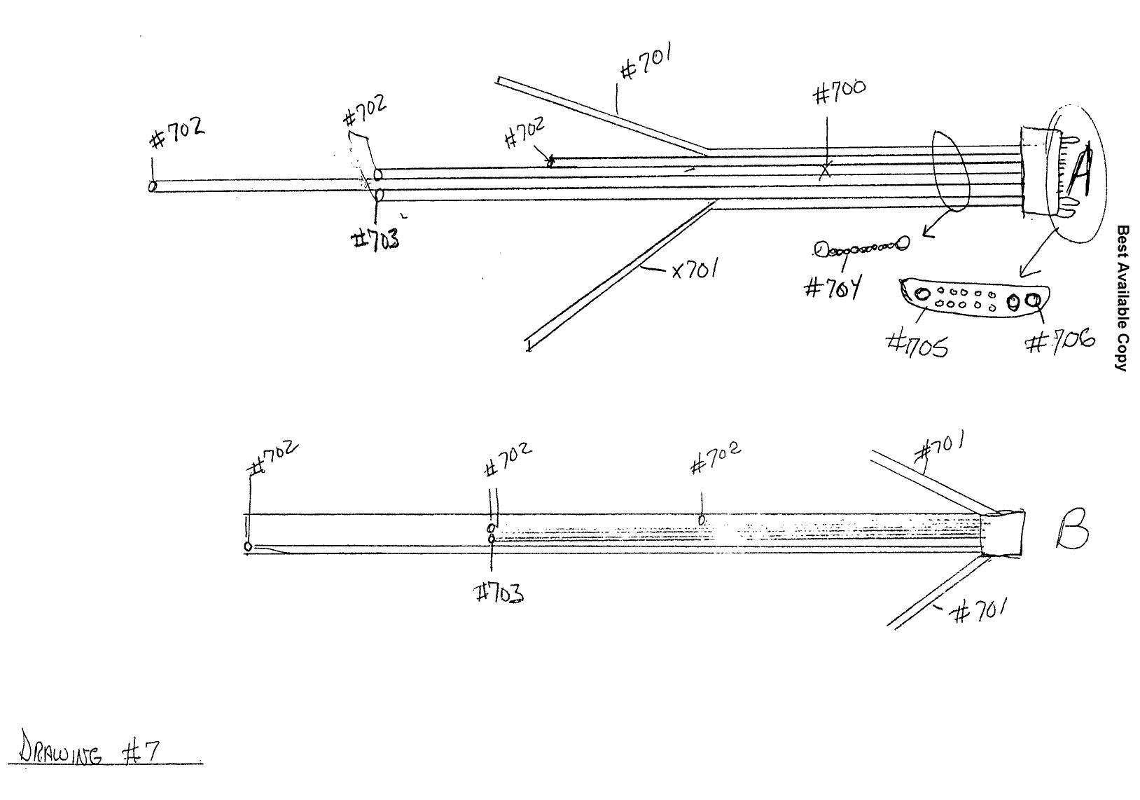

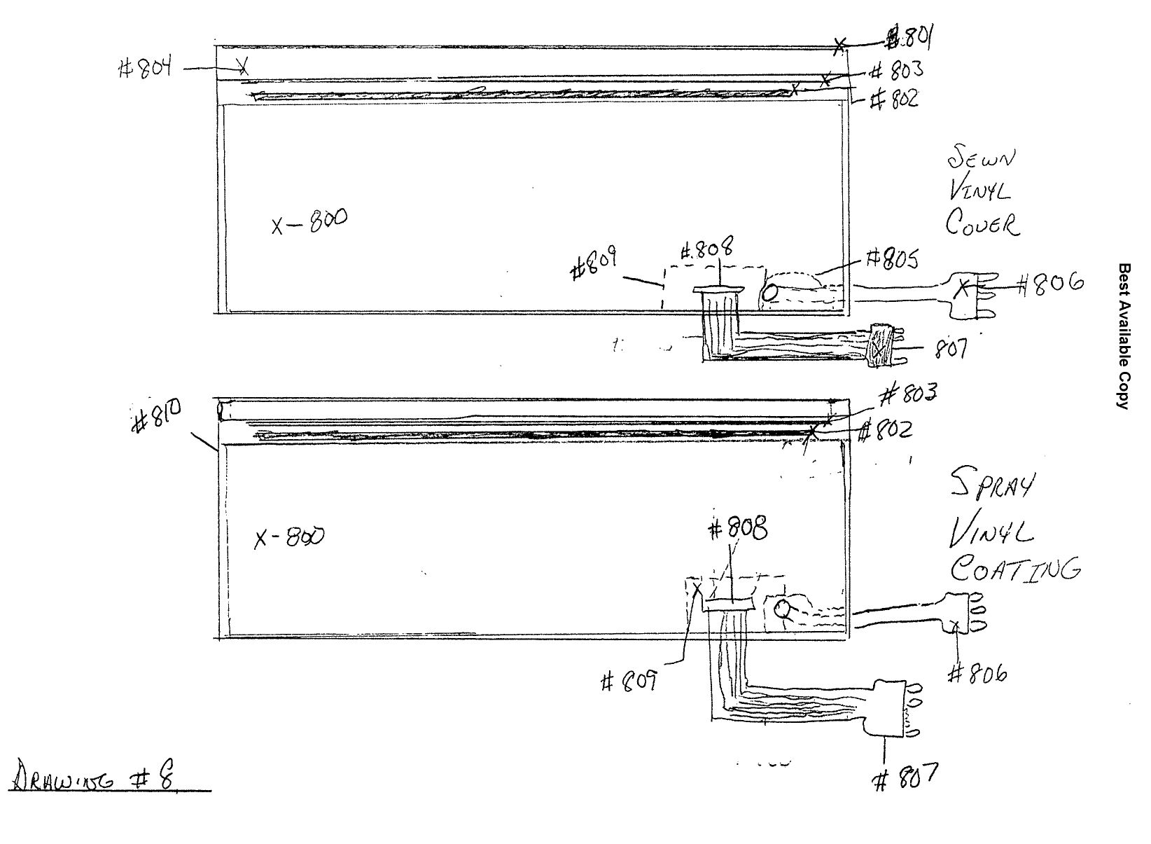

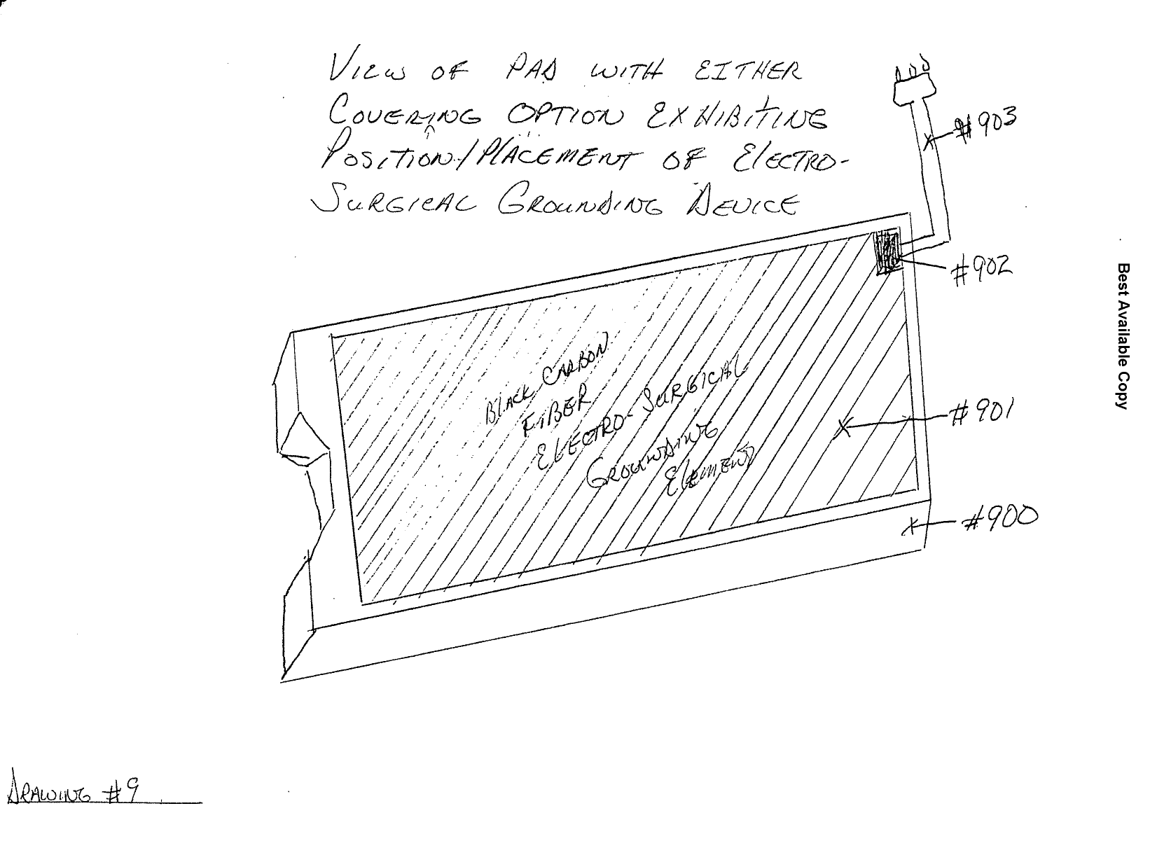

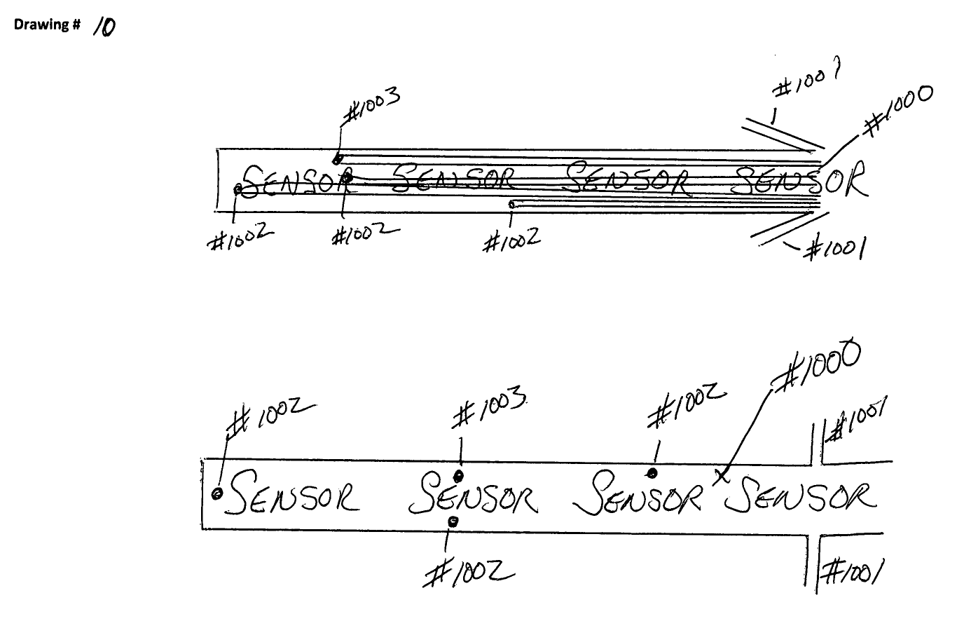

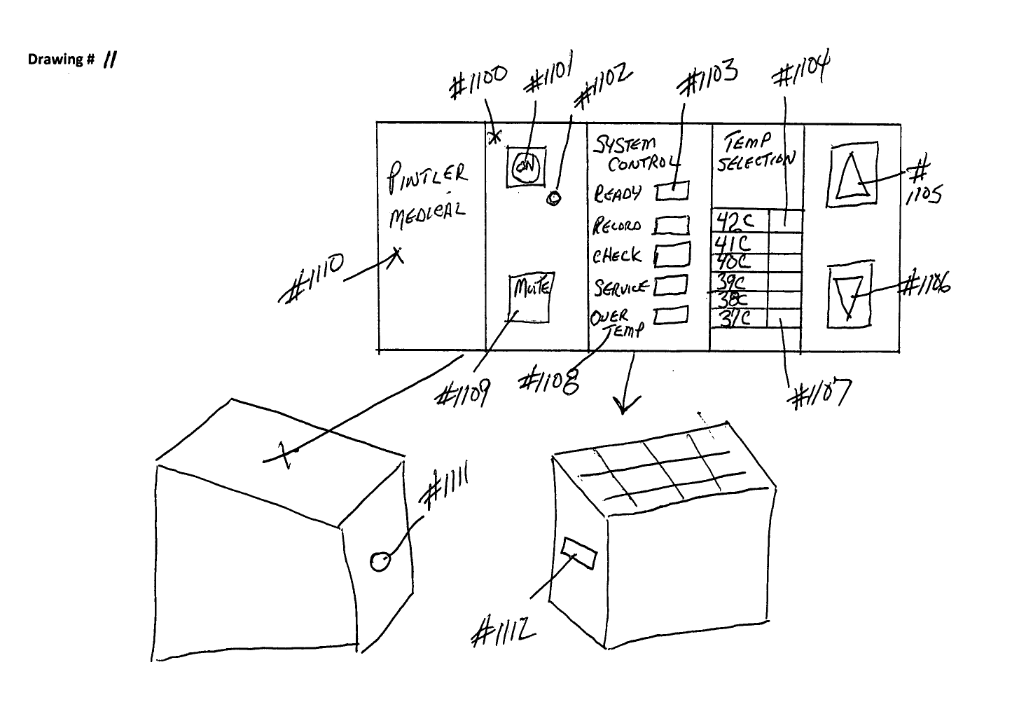

Drawing #3 embodiment of the resistive element 300 and electro-surgical grounding device 304 are shown as positioned length and width in a standard operating room table pad 100. This embodiment exhibits the “centered” relationship of the temperature measurement system 101/301 to the pad's overall width/length. The center/median relation is important to the patient as 99% of all patients, pediatrics-obese, are center positioned on the operating table pad. Patients may be positioned higher or closer to the head section 110 or moved lower on the pad and closer to the lower leg section 109, but will centered. In this embodiment the resistive element 300 and electro-surgical grounding device 304 exhibit a contact and close proximity relationship beneath the pad's optional covers, sewn vinyl/spray coat vinyl. Exhibited in FIG. 3 is the position of exit cables/ribbons 302/303 from the standard operating table pad 100. In this embodiment the pad exhibits a side zipper 305 identified for ease of assembly and access for removal or service of the electro-surgical capacitor element.

Drawing #4 exhibits a lateral view of the pad's end. Cables/ribbons 410/411 exit the pad. The resistive heating element 402 is exhibited as a topical layer to the pad's foam base 401. The electro-surgical grounding device 403 is shown as positioned upon or above the resistive heating element 402, but these two products can be reversed, whereas 403 could be positioned beneath 402. This placement decision depends on the surgeon's comfort levels of making adjustments to 115 the electro-surgical generator's power control selections. Physician training and articles heavily influence the settings of 115 the electro-surgical generator.

Drawing #4 exhibits 411 the cable/ribbon traversing beneath 400 the pad's cover material and guiding to 101 the center channel/sheath of the pad. 404 exhibits the 18 AVG wires guiding to 402 the resistive heating element to mate w/602 connection to the element. Position upon the base foam material would be a layered devices: 401 the resistive heating element, 403 the electro-surgical grounding device, 600 temperature management system and 405 the thermal diffusion material. 405 thermal transfer felt material exhibits the characteristics of thermal passage versus insulation characteristics and is positioned 400 the topical cover. 405 thermal transfer material can be positioned upon or below the 403 electro-surgical capacitor element.

Drawing #5 exhibits the embodiment of 402 the heating element with details of design and construction. In this embodiment 500 is identified as the buss bar to deliver electrical current to the resistive element. The embodiment 501 reveals bars of resistive element attached to 500 the buss bars. Embodiment 502 identifies the spacing or open space between 501 resistive bars. In this embodiment 502 open spacing between the resistive bars allows 403 to be positioned above or beneath 402 the heating element. 504 reveals the heating elements surface exceeds the buss bar position.

Drawing #6 reveals the embodiment 402 the resistive heating element and the connection to 104 the control unit via 102 the cable/ribbon. In the embodiment 504/601 18 AVG wiring leads from 102 cable/ribbon are crimped/mated at 602 the tubular connection site of 603 the Tyco crimp power connection site. Embodiment 603 reveals a crimp connector designed to perforate 500/605 buss bar material that exhibits a protected laminate coating. In this embodiment the positioning of 600/700 the temperature management system (s) to 402 heating element is revealed. In the embodiment 600/700 the temperature management system is positioned upon or beneath 402 the heating element, in a position parallel and close proximity to 500 the buss bar.

Drawing #7 reveals the embodiment 700 the temperature measurement cable/ribbon (s). 702/703 reveals the mounting of RTD thermister sensors upon either 700 a Cicoil type ribbon/cable or a Minco Flex Circuit ribbon/cable. In the embodiment both designs reveal 601/701 splintering from 600/700 the ribbon/cable that joins 100 the heated/grounding pad and unit 104/203 control unit. Exhibited in the embodiment is 705 the use of a Sub “D” connector with a 706 pin aligned specifically designed and selected to prevent operator connection misalignment.

Drawing #8 exhibits an embodiment whereas the selection of pad finishes (sewn vinyl or spray coat vinyl) exhibit two of exit sites 808 for the ribbon/cable and 805 for the electro-surgical capacitor element. 100 the heated/grounding pad connection cable ribbon mates to 104/203 control unit and 103/211/303/410/805 reveal the electro-surgical grounding device cable exits the pad to connect to the electro-surgical generator. This figure reflects the two types of pad exhibiting slightly different position of cable exits 808/805 but performing the same function in either cover selection, exit of cables from 800 the pad.

Drawing #9 embodiment 900 identifies an OR pad supporting 901 a black carbon fiber electro-surgical grounding element or device. Embodiment 902 reveals the site where the black carbon fiber element and 103/211/303/410/805 join and exit 100 the operating room pad. This embodiment identifies 901 black carbon fiber device as the electro-surgical grounding element capable of being positioned in three modes: 1) topical or upon 402 the heating element and beneath the outer covering material fabric or finish. 2) Beneath 402 the heating element and 405 thermal diffusing material. 3) Positioned upon the heating element 402 beneath 405 the thermal diffusion layer

Drawing #10 embodiment reveals added assembly of 600/700/1000 the temperature measurement ribbon/cables. The embodiment 1004 reveals a labeling beneath 600/700/1000 the Cicoil ribbon or Minco Flex Circuit exhibiting the words “sensor-sensor-sensor” in a non-Xray transparent material. The design offers the operator a clear identity of sensors wiring and RTD thermisters to reduce mistaken identity of material during X-ray events.

Drawing #11 embodiment 1100 reveals the operator's control membrane. Embodiments 1101 reflects the operator's Standby/ON button. 1102 LED identifies unit has power and reveals yellow LED when unit has power. 1103 LED operational information, LED's reveal Ready to function or service information. 1104 exhibits the temperature selection bar, a sequence of LED's that light when a temp is selected and remains on as the next higher selection is chosen. When temperature is lowered the LED's descend and extinguish. 1105/1106 temperature selection arrows for raising or lower the units thermal output. 1105/1106 perform dual functions, they can be used to turn the unit on from Standby. 1106 when activated with 1109 jointly allows the operator to perform a LED light and audible alarm check. 1107 reflects the LED light bar for sequential temperature selection. 1108 reveals service information to the operator. 1109 functions as an alarm mute button in the event the audible alarm is activated. 1110 exhibits the Pintler Medical company identity label. 1111 identifies the site of the pass through temperature cable connection for joining 106 the patient monitoring cable w/patient monitor and the device. 1112 reveals the site and position of the Sub D connector 412/600/806/807/1000.

1. An apparatus such as a surgical operating table pad which provides a method to warm patients during surgery and/or function as grounding surface for the electro-surgical cautery current generated by the electro-surgical generator. The apparatus has two components:
a surgical table/clinical diagnostic pad, and a control unit with microprocessor for specific operational software/firmware.
The function of the surgical/diagnostic pad is dual purpose: 1) offering the operator an electro surgical grounding capacitor to complete the loop or cycle generated by the electro-surgical generator, 2) a patient warming surface. A cable/ribbon connects the resistive heating element to a power unit containing a CPU board and microprocessor for the operation and control of resistive element heating temperatures. A separate cable joins the capacitor element to a electro-surgical generator positioned separately from the pad's control unit. These two elements can operate simultaneously or independently, patient warming and/or electro surgical grounding cycling current from the electro-surgical cautery device and generator.

2. The pad's cover can be either a vinyl cover or a spray coat vinyl cover.

3. The cover in claim #2 can be a vinyl material such as Vintex, Canada.

4. The covering in claim #2 can be a spray coat vinyl, such as available from PlastiDip, Minnesota.

5. The operating room table pad cover materials of claim #2 will exhibit an entry site/port for the passage of cable (s) that is resistant to fluid ingression and a restraint for the tug/pull upon the cable/ribbon connecting pad and power unit.

6. The vinyl operating table pad of claim #2 exhibits a fluid resistant pocket/sheath/channel thermally welded and sealed to the pad's outer covering fabric. The pocket/sheath/channel is visible to the operator and identified as temperature sensor channel.

7. The vinyl covering fabric of claim #2 can be so designed as to allow the removal of the electro surgical grounding capacitor device of claim #1 via a pocket or utilization of a zipper access.

8. The operating table pad device of claim #1, embraces, encloses, covers, protects from exposure and can embed a predetermined electrical conductivity element such as Black Carbon Fiber element/device to function as a “electro-surgical grounding device” connecting via cable to the electro-surgical generator.

9. The electrical conductivity element of claims #8 is compatible with electrosurgical generators when an adaptor is utilized to compliment plug configurations, i.e. Valley Lab, ERBE, Megadyne, Arrow and others.

10. The electrical conductivity element of claim #8 exhibits a size between 200 & 1200 square inches.

11. The electrical conductivity element of claim #8 will be invisible to X-ray imagining

12. The electrical conductive element of claim #8 is independent of the heating element and thermal diffuser material.

13. The operating room table pad design of claims #1, #2, & #6 are so designed as to allow the positioning of a flat circuit/ribbon cable such as a Flex Circuit/Thermal Ribbon Strip, Minco, Minn. or ribbon cable, Cicoil, Valencia, Calif.

14. Positioned/soldered upon or attached to the flat circuit/ribbon cable of claim #13 will be numerous RTD thermister sensors at a variety of locations. The sensors will be the type such as Spectrum Sensors and Control, A1004 C-C3 (10,000 mili-ohms) & A2253-C3 (3,000 mili-ohms). This system will be identified as the temperature measurement system

15. The temperature measurement system of claim (s) 13/14 can be positioned within the temperature channel of claim #6.

16. The temperature system can be positioned upon or below the heating element of claim #21.

17. The thermister sensors of claim #14 share a common excitation.

18. The temperature measurement system of claim (s) No. 13/14 can exhibit a covering/coating/foil sleeve or flat surface to identify the temperature management system to diagnostic radiology that does produce an X-ray visible image.

19. The device in claim #18 can exhibit printing or wording such as: sensor-sensor-Pintler-sensor-etc. Other words or languages can be utilized for this identification, such as: patient-patient-patient, wire-wire-wire, or a script of any imaginable wording that aids in X-ray identity.

20. The resistive heating element of claim #1 is low voltage, 48V, 80 W per meter². Element is so designed as to appear as a ladder with gaps between the heating bars. Such as the type provided by Ebecco, Company, United Kingdom.

21. The resistive element of claim #20 exhibits connection terminals (2) of the type by Tyco Co., Type 3, Foil Conductor Terminal #52584.

22. The heating element of claim #20 is adhesively joined to a layer of material identified as a thermal diffusion material: a product such Ecofelt Equinox Polyester from Aetna Felt Corp, Allentown, Pa. or a layer of visco-elastic foam such as available from FXI/Foamex, Auburn, Ind.

23. The control unit of claim #1 is a metal encasement (aluminum/steel) which houses the circuit board, microprocessors, connection ports, wireless transmission site, mounting clamp for IV pole attachment, and operator's control & information membrane.

24. The control unit of claim #1 can be embedded and so designed as to be a component of the operating table's physical design, structure, power or electrical system (s).

25. The operator's control overlay membrane of the control unit claim #23 advises the operator of operational data via LED indicator lights regarding patient warming functionality.

26. The operator's control in the embodiment of claim #25 can be a component of the operating table's hand controller for ambulation of the table's surface or an independent temperature information and control device or integrated into the

27. The control unit microprocessor software of claim #27/#28 methods allow temperature selection exhibit control in Celsius: 37 C, 38 C, 39 C, 40 C, 41 C and 42 C, utilizing LED light indicators to advise the operator of selection and setting.

28. The microprocessor software of claim #23/#24 methods exhibit a programmed maximum temperature selection of 41.7 C, selection of 42 C by the operator is programmed at 41.7 C.

29. The microprocessor software of claim #23/#24 methods exhibit the ability to monitor each thermister of claim #14 and control power to the resistive heating element based on the highest temperature value measured by a single thermister of claim #14.

30. The control unit microprocessor software of claim #23/#24 methods respond to temperature input from the sensors of claim #16 and cycles power to the resistive heating element of claim #20 to maintain a steady state of the temperature selected by the operator.

31. The control unit microprocessor's software of claim #23/#24 methods allow the operator to verify the audible warning system with a dual action: selecting two control buttons and activating them simultaneously to test the functionality of the audible alarm and all operational LED's.

32. The control unit microprocessor's software of claim #23/#24 method of transferring from Standby to ON can occur via two (2) optional command actions: Pressing ON/Standby button or Pressing either the UP or DOWN arrows of temperature selection.

33. The control unit microprocessor's software of claim #23/#24 methods will report “Over-Temp” and execute Over-Temp safety functions if the temperature measurement system of claim #14 reports a temperature at any sensor 2 C above the operator's selected temperature, i.e. operator sets device at 41 C, after achieving 41 C then elects to a lower setting of 38 C, the temperature variance executes safety and alerts the operator of a +2 C variance.

34. The control unit microprocessor's software of claim #23/#24 will activate an audible alarm positioned on the circuit board if an operator attempts to turn the unit ON while the unit is in an Over-Temp/Power Off safety mode.

35. The control unit microprocessor's of claim #23/#24 exhibits two circuit boards, one analog and one digital.

36. The control unit of claim #23/#24 exhibit the capability to perform wireless modes.

37. The control unit claim #23/#24 exhibits a connector port for the attachment of a cable mating patient monitoring system/electronic record or performing this function wirelessly.
